(19) United States
(12) Patent Application Publication
(10) Pub. No.: US 2020/0375877 A1
(43) Pub. Date: Dec. 3, 2020

(54) APPARATUS, METHODS AND SYSTEMS FOR LOW ENERGY PROCESSING OF CONSUMER PERSONAL HYGIENE PRODUCTS; FORMULATIONS OF THE SAME

(71) Applicant: Act + Acre, Inc., New York, NY (US)

(72) Inventor: Colm MACKIN, New York, NY (US)

(21) Appl. No.: 16/996,119

(22) Filed: Aug. 18, 2020

Related U.S. Application Data
(63) Continuation of application No. PCT/US2019/019013, filed on Feb. 21, 2019.
(60) Provisional application No. 62/633,312, filed on Feb. 21, 2018.

(51) Int. Cl. A61K 8/92; A61Q 5/02; A61Q 5/12; A61K 8/46; A61K 8/44; A61K 8/60; A61K 8/42; A61K 8/81; A61K 8/34; A61K 8/37; A61K 8/19; A61K 8/49; A61K 8/73

(52) U.S. Cl. CPC A61K 8/922; A61K 2800/596; A61Q 5/12; A61K 8/466; A61K 8/44; A61K 8/608; A61K 8/442; A61K 8/42; A61K 8/8152; A61K 8/345; A61K 8/375; A61K 8/19; A61K 8/494; A61K 8/737; A61K 8/347; A61K 8/37; A61K 8/732; A61Q 5/02

(57) ABSTRACT

Embodiments described herein relate generally to apparatus, methods and systems for low energy processing of consumer personal hygiene products (PHPs) and formulations of the same. In some embodiments, a method of manufacturing a consumer PHP comprises combining a first material and second material at room temperature to form a first mixture, mixing the first mixture at room temperature, adding a third material at room temperature to form a second mixture; mixing the second mixture at room temperature, adding a fourth material at room temperature to form a third mixture; and mixing the third mixture at room temperature. In some embodiments, the third material and the fourth material can be mixed to form the second mixture and the first mixture and second mixture can be combined to form the third mixture. In some embodiments, the mixing can be accomplished using a propeller mixer. In some embodiments, the propeller mixer can be rotated at a speed of greater than about 200 rpm during at least one of the mixing steps. In some embodiments, the pH of the third mixture can be adjusted to between about 4.5 and about 6.5 using an acidifier. In some embodiments, the viscosity of the third mixture can be adjusted to between about 100 cP and about 25,000 cP using a thickener.

This application claims priority to and the benefit of U.S. Provisional App. No. 62/633,312, filed Feb. 21, 2018; the entirety of the aforementioned application is herein expressly incorporated by reference for all purposes. This application for letters patent disclosure document describes inventive aspects that comprise various novel innovations (hereinafter “disclosure”) and contains material that is subject to copyright, mask work, and/or other intellectual property protection. The respective owners of such intellectual property have no objection to the facsimile reproduction of the disclosure by anyone as it appears in published Patent Office file/records, but otherwise reserve all rights.

### FIELD

The present innovations generally address apparatuses, methods, and systems for personal hygiene product manufacturing, and more particularly, APPARATUS, METHODS AND SYSTEMS FOR LOW ENERGY PROCESSING OF CONSUMER PERSONAL HYGIENE PRODUCTS; FORMULATIONS OF THE SAME.

### BACKGROUND

Conventional consumer personal hygiene products, such as shampoos, gels, and conditioners, are typically manufactured in a batch-wise compounding process at large volumes, for example volumes greater than about 3,000 U.S. gallons. Heating the compositions during the compounding process facilitates mixing and activation of various components.

### SUMMARY

Embodiments described herein relate generally to apparatus, methods and systems for low energy manufacturing and processing of consumer personal hygiene products (PHPs) and formulations of the same. Embodiments described herein are discussed with reference to methods of manufacturing PHPs using less energy than conventional manufacturing methods, thereby reducing the environmental impact (e.g., providing a PHP with a reduced “carbon footprint” relative to other PHP manufactured by traditional energy-intensive methods). In some embodiments, a method of manufacturing a consumer PHP comprises combining a first material and second material at a particular environmental energy state or range (e.g., ambient temperature/pressure, Normal Temperature and Pressure—20° C./293.15 K/68° F. at an absolute pressure of 1 atm/14.696 psi/101.325 kPa—and/or ranges based thereon, colloquially “room temperature”, which is used by way of illustration not limitation) to form a first mixture, further mixing the first mixture at room temperature, adding a third material to the first mixture at room temperature to form a second mixture; further mixing of the second mixture at room temperature, adding a fourth material to the second mixture at room temperature to form a third mixture; and further mixing of the third mixture at room temperature. In some embodiments, the third material and the fourth material can be mixed to form the second mixture and the first mixture and second mixture can be combined to form the third mixture. In some embodiments, the mixing can be accomplished using a propeller mixer. In some embodiments, the propeller mixer can be rotated at a speed of greater than about 200 rpm during at least one of the mixing steps. In some embodiments, the pH of the third mixture can be adjusted to between about 4.5 and about 6.5 using an acidifier. In some embodiments, the viscosity of the third mixture can be adjusted to between about 100 cP and about 25,000 cP using a thickener. According to some embodiments, a cold process method of manufacture is disclosed. It is to be understood that, as used herein, the disclosure is directed to method and apparatuses where heat energy (i.e., thermal, kinetic, etc.) is reduced, substantially reduced, substantially eliminated, or eliminated. In such embodiments, the lack of added energy (i.e., added heat) reduces or eliminates loss of small molecule compounds that would otherwise be released by mixing with added energy. Some embodiments can utilize sensors to reduce added energy (i.e., slow mixing if temperature of a mixture exceeds a given threshold and/or relative to ambient).

According to some embodiments, a personal hygiene product composition is disclosed, comprising: (1) a first material comprising at least two selected from: sodium lauroyl methyl isethionate, PEG-150 pentaerythrityl tetrastearate, sodium cocoyl apple amino acids, polysorbate 20, lauramidopropyl betaine, water, hydroxypropyl starch phosphate, caprylic/capric/myristic/stearic triglyceride, glycerin, parfum, isoamyl laurate, caprylic/capric triglyceride, hydrogenated vegetable oil, polysorbate 60, trimethylolpropane tricaprylate/tricaprate, acetamidopropyl trimonium chloride, C12-C15 alkyl benzoates, isostearyl linoleate, sunflower oil, and soybean oil; (2) a second material comprising at least two from among parfum, glycerin, PPG-2 hydroxyethyl cocamide, acrylates copolymer, adansonia digitata seed oil, amaranthus caudatus seed oil, cananga odorata flower oil, caprylyl glycol, cocamide mipa, disodium edta, glycol distearate, Helianthus annuus seed oil, hexylene glycol, Lavandula angustifolia oil, menthyl ethylamido oxalate, moringa oleifera seed oil, panthenol, phenoxyethanol, potassium sorbate, Rosmarinus officinalis leaf extract, adansonia digitata seed oil, amaranthus caudatus seed oil, cananga odorata flower oil, caprylyl glycol, cetrimonium chloride, citric acid, Crambe abyssinica seed oil, decyl glucoside, dicaprylyl ether, disodium edta, ethylhexyl olivate, glyceryl oleate, guar hydroxypropyltrimonium chloride, hexylene glycol, hydrogenated castor oil/sebacic acid copolymer, hydrolyzed silk, hydrolyzed soy protein, isostearyl linoleate, sunflower oil, soybean oil, aloe, and calendula in sunflower oil; (3) a third material comprising at least two from among sodium hydroxide, sodium lauroamphoacetate, steareth-4, xanthan gum, benzyl alcohol, benzyl benzoate, benzyl cinnamate, benzyl salicylate, cinnamal, citral, eugenol, farnesol, geraniol, isoeugenol, limonene, linaloolis, hydroxyethylcellulose, Lavandula angustifolia oil, moringa oleifera seed oil, palmitamidopropyltrimonium chloride, panthenol, Passiflora edulis seed oil, phenoxyethanol, polyquaternium-37, potassium sorbate, PPG-1 trideceth-6, propylene glycol dicaprylate/dicaprate, Rosmarinus officinalis leaf extract, sodium hydroxide, sorbitan oleate, squalene, benzyl alcohol, benzyl benzoate, benzyl cinnamate, benzyl salicylate, cinnamal, citral, eugenol, farnesol, geraniol, isoeugenol, limonene, linalool, aloe, calendula in sunflower oil, vetiver oil, orange extract, ylang oil, moringa oleifera, adasonia digitata seed, and amaranthus caudatus seed oil; and (4) a fourth material comprising at least two from among myristamidopropyl betaine, sodium benzoate, tetrasodium glutamate diacetate, trisodium ethylenediamine disuccinate, benzoic acid, benzyl alcohol, propylene glycol, sodium benzoate, vetiver oil, orange extract, ylang oil, moringa oleifera, adasonia digitata seed, amaranthus caudatus seed oil, and basil root extract, wherein substantially no thermal energy is added to the first material, the second material, the third material, and the fourth material during a compounding process. As disclosed herein, the statement “substantially no thermally energy added” means that the materials are not heated (e.g., not heated with heaters or by introduction of hot materials, air, gas, etc. to the mixture, and/or that heat added via kinetic energy (e.g., from mixing) does not change the temperature of the mixture by more than about 15 degrees C., 12 degrees C., 10 degrees C., 9 degrees C., 8 degrees C., 7 degrees C., 6 degrees C., 5 degrees C., 4 degrees C., 3 degrees C., 2 degrees C., or 1 degree C. According to some embodiments, a method of manufacturing a personal hygiene product, comprises: combining a first material and second material at room temperature to form a first mixture; mixing the first mixture at room temperature; combining a third material and the first mixture at room temperature to form a second mixture; mixing the second mixture at room temperature; combining a fourth material and the second mixture at room temperature to form a third mixture; and mixing the third mixture at room temperature.

### BRIEF DESCRIPTION OF THE DRAWINGS

The accompanying appendices, drawings, figures, images, etc. illustrate various example, non-limiting, inventive aspects, embodiments, and features (“e.g.,” or “example(s)”) in accordance with the present disclosure:

FIG. 1 is a schematic flow diagram of a method for preparing a consumer hygiene product, according to an embodiment.

### DETAILED DESCRIPTION

Embodiments described herein relate generally to methods of manufacturing consumer personal hygiene products (PHPs) using less energy than conventional manufacturing methods. Embodiments described herein also relate generally to the consumer PHPs manufactured using a manufacturing process that results in less cost and environmental impact associated with manufacturing.

Conventional PHPs can comprise products such as hair shampoos, gels, skin moisturizers, detoxifying solutions, hand lotions scalp detoxifiers, and hair conditioners. PHPs are typically used to cleanse, moisturize, condition, detoxify, or in other ways improve the health or aesthetic qualities of hair and/or skin when applied. Over the past thirty years or so, PHPs have become significantly more complex to manufacture because these products often now comprise tens or even hundreds of ingredients. PHPs are often somewhat viscous formulations, often water-based, and often comprise detergents, foam boosters, thickeners, conditioning agents, preservatives, rheology modifiers, surfactants, gelling agents, colorants, odorants, fragrance oils, stabilizers, opacifiers, and/or other additives.

Conventional PHPs are typically manufactured in a complex, batch-wise compounding process at volumes of greater than about 3,000 U.S. gallons. The complexity of manufacturing PHPs arises not only because each of the long list of ingredients can react chemically with any of the other ingredients, but also because mixing the ingredients can often necessitate heating and/or high-speed mixing to homogenize the resulting product. In order to be able to mix together the complex array of ingredients typically found in PHPs, conventional PHP manufacturing processes often use heat during compounding to help raw materials combine more quickly. The conventional compounding process can often take between about one hour and about five hours to achieve a homogenous mixture and the admixtures and formulations can reach temperatures of 50° C. to greater than 70° C. Conventional PHPs typically contain harmful surfactants such as sodium lauryl sulfate and/or sodium laureth sulfate that can interact and/or combine with other ingredients at these elevated temperatures and over time to form nitrosamines, which are known human carcinogens and can lead to human kidney and respiratory damage based upon exposure pathway. Conventional PHPs also often contain silicone and parabens, which are often used as conditioners and preservatives, respectively, and which are likely carcinogens, endocrine disruptors, and/or may exhibit reproductive toxicity. During this conventional heated compounding and mixing process, one or more of the raw materials can be chemically and/or physically altered in such a way so as to reduce the efficacy or quality of the raw material. Without wishing to be bound by any particular theory, the chemical and/or physical alteration of raw materials in the compounded PHP product can result in a finished product that has a reduced hygienic effect such that the PHP does not effectively clean the hair and/or skin as well, or the resulting PHP can remove more sebum than desired, can strip the hair and/or skin of nutrients, and other deleterious effects.

The increased formulation temperature during conventional compounding of PHPs can result in the degradation of many natural ingredients such as have been used for many decades in PHPs. This means many conventional PHP manufacturers use more thermally resistant ingredients that can represent a toxicological risk, can be dermatologically harmful, and/or are more harmful to the environmental based upon fate and transport of the PHP or degradants thereof. Heating large fluid volumes during the PHP compounding process requires a significant amount of energy, both for the heating and the increased intensity of mixing, which increases the financial cost and environmental impact of manufacturing.

Thus, it is an enduring goal of consumer personal hygiene product manufacturers to reduce the cost and environmental impact of the manufacturing process.

It has surprisingly been found that manufacturing PHPs according to the embodiments described herein without heating the formulations and admixtures does not compromise the homogeneity of the formulation nor does it reduce the efficacy of the finished PHPs. It has also surprisingly been found that manufacturing PHPs without heating the formulations and admixtures enables the use of natural and/or more therapeutically efficacious ingredients that are otherwise thermally degrading according to conventional manufacturing methods.

In some embodiments, the present invention provides compositions for topical and/or transdermal delivery, e.g., as a cream, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, conditioners, detoxification ointments, scalp detoxifiers, or soaps, or dermatological compositions such as lotions, ointments, gels, creams, liniments, patches, deodorants, or sprays.

In some embodiments, provided compositions are stable for extended periods of time, such as greater than 1 week, greater than 2 weeks, greater than 1 month, greater than 2 months, greater than 6 months, greater than 1 year, greater than 2 years, greater than 3 years, or more.

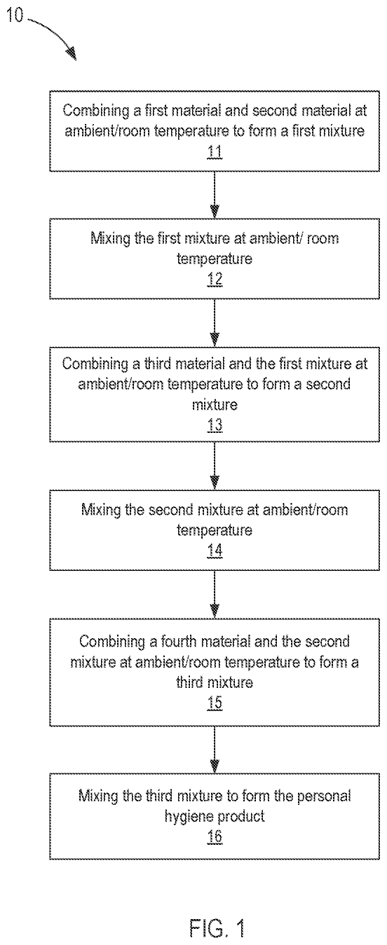

FIG. 1 illustrates a method of manufacturing a PHP 10 that uses less energy for heating ingredients during mixing, comprising combining a first material and a second material at room temperature to form a first mixture 11.

In some embodiments, combining the first material and the second material to form the first mixture 11 can comprise mixing the first material into the second material and/or mixing the first material and second material together with solvent (e.g., water) to facilitate mixing of the first and second materials. In some embodiments, the first material can be a different state of matter than the second material. For example, the first material can be a solid (e.g., a powder) while the second material can be a liquid (e.g., water). In some embodiments, the first and second material can be at least one of a liquid, a gas, or a solid. In some embodiment, the first material can be communicated into a mixing vessel containing the second material. Alternatively, in some embodiments, the first material and the second material can be communicated together into a new mixing vessel, e.g., by merging the two flow paths. In some embodiments, the first material and the second material can be at least partially mixed together before being communicated into the new mixing vessel. In some embodiments, this pre-mixing of the first material and the second material can be accomplished using a static (in-line) mixer, via inherent turbulence due to flow path tortuosity, through axial mixing, through longitudinal mixing, through laminar flow turbulence, using a Tee Mixer, using a coaxial jet mixer, or using any other suitable in-pipe mixing methods.

In some embodiments, the first material can be a mixture of materials comprising at least two of water, sodium lauroyl methyl isethionate, peg-150 pentaerythrityl tetrastearate, sodium cocoyl apple amino acids, polysorbate 20, lauramidopropyl betaine, hydroxypropyl starch phosphate, caprylic/capric/myristic/stearic triglyceride, glycerin, fragrance (parfum), isoamyl laurate, caprylic/capric triglyceride, hydrogenated vegetable oil, polysorbate 60, trimethylolpropane tricaprylate/tricaprate, and acetamidopropyl trimonium chloride, C12-C15 alkyl benzoate, and/or isostearyl linoleate.

In some embodiments, the first material can be between about 0.01 wt % and about 99 wt % of the finished formulation, between about 1 wt % and about 90 wt %, between about 2 wt % and about 85 wt %, between about 3 wt % and about 80 wt %, between about 4 wt % and about 75 wt %, between about 5 wt % and about 70 wt %, between about 6 wt % and about 65 wt %, between about 7 wt % and about 60 wt %, between about 8 wt % and about 55 wt %, between about 9 wt % and about 50 wt %, between about 10 wt % and about 45 wt %, between about 11 wt % and about 40 wt %, between about 12 wt % and about 35 wt %, between about 13 wt % and about 30 wt %, between about 14 wt % and about 25 wt %, between about 15 wt % and about 20 wt %, between about 1 wt % and about 50 wt %, between about 1 wt % and about 25 wt %, between about 1 wt % and about 20 wt %, between about 20 wt % and about 99 wt %, between about 25 wt % and about 98 wt %, between about 30 wt % and about 97 wt %, between about 35 wt % and about 96 wt %, between about 40 wt % and about 95 wt %, between about 45 wt % and about 94 wt %, between about 50 wt % and about 93 wt %, between about 55 wt % and about 92 wt %, between about 60 wt % and about 91 wt %, between about 65 wt % and about 90 wt %, between about 70 wt % and about 85 wt %, and about 75 wt % to about 85 wt %, inclusive of all values and ranges therebetween.

In some embodiments, the second material can be a mixture of materials comprising at least two of fragrange (parfum) glycerine, ppg-2 hydroxyethyl cocamide, acrylates copolymer, adansonia digitata seed oil, amaranthus caudatus seed oil, canaga odorata flower oil, caprylyl glycol, cocamide mipa, disodium edta, glycol distearate, Helianthus annuus (sunflower) seed oil, hexylene glycol, Lavandula angustifolia (lavender) oil, menthyl ethylamido oxalate, moringa oleifera seed oil, panthenol, phenoxyethanol, potassium sorbate, Rosmarinus officinalis (rosemary) leaf extract, adansonia digitata seed oil, amaranthus caudatus seed oil, cananga odorata flower oil, caprylyl glycol, cetrimonium chloride, citric acid, Crambe abyssinica seed oil, decyl glucoside, dicaprylyl ether, disodium edta, ethylhexyl olivate, glyceryl oleate, guar hydroxypropyltrimonium chloride, Helianthus annuus (sunflower) seed oil, hexylene glycol, hydrogenated castor oil/sebacic acid copolymer, hydrolyzed silk, hydrolyzed soy protein, sunflower oil, soybean oil, aloe, calendula in sunflower oil, vetiver oil, orange oil, ylang oil, moringa oleifera, adasonia digitata seed, and/or amaranthus caudatus seed oils.

In some embodiments, the second material can be between about 0.01 wt % and about 99 wt % of the finished formulation, between about 1 wt % and about 90 wt %, between about 2 wt % and about 85 wt %, between about 3 wt % and about 80 wt %, between about 4 wt % and about 75 wt %, between about 5 wt % and about 70 wt %, between about 6 wt % and about 65 wt %, between about 7 wt % and about 60 wt %, between about 8 wt % and about 55 wt %, between about 9 wt % and about 50 wt %, between about 10 wt % and about 45 wt %, between about 11 wt % and about 40 wt %, between about 12 wt % and about 35 wt %, between about 13 wt % and about 30 wt %, between about 14 wt % and about 25 wt %, between about 15 wt % and about 20 wt %, between about 1 wt % and about 50 wt %, between about 1 wt % and about 25 wt %, between about 1 wt % and about 20 wt %, between about 20 wt % and about 99 wt %, between about 25 wt % and about 98 wt %, between about 30 wt % and about 97 wt %, between about 35 wt % and about 96 wt %, between about 40 wt % and about 95 wt %, between about 45 wt % and about 94 wt %, between about 50 wt % and about 93 wt %, between about 55 wt % and about 92 wt %, between about 60 wt % and about 91 wt %, between about 65 wt % and about 90 wt %, between about 70 wt % and about 85 wt %, and about 75 wt % to about 85 wt %, inclusive of all values and ranges therebetween.

In some embodiments, combining the first material and the second material to form the first mixture 11 can comprise combining the first material and the second material (or the mixtures of materials formed therefrom) instantaneously or at a metered rate while stirring or mixing the admixture. In some embodiments, the second material can be added to the first material at a rate of less than about 80 wt % per minute, less than about 70 wt % per minute, less than about 60 wt % per minute, less than about 50 wt % per minute, less than about 40 wt % per minute, less than about 40 wt % per minute, less than about 30 wt % per minute, less than about 20 wt % per minute, less than about 15 wt % per minute, less than about 12 wt % per minute, less than about 10 wt % per minute, less than about 8 wt % per minute, less than about 6 wt % per minute, less than about 5 wt % per minute, less than about 4 wt % per minute, less than about 3 wt % per minute, less than about 2 wt % per minute, or less than about 1 wt % per minute, inclusive of all values and ranges therebetween. In some embodiments, the rate at which the admixture is stirred during the combining 11 step can be less than about 300 rpm, less than about 280 rpm, less than about 260 rpm, less than about 240 rpm, less than about 220 rpm, less than about 200 rpm, less than about 180 rpm, less than about 160 rpm, less than about 140 rpm, less than about 120 rpm, less than about 100 rpm, less than about 80 rpm, less than about 60 rpm, less than about 40 rpm, or less than about 20 rpm, inclusive of all values and ranges therebetween.

In some embodiments, the temperatures of the first material, the second material, and/or the first mixture can be maintained at about room temperature. In some embodiments, the temperatures of the first material, the second material, and/or the first mixture can be maintained at less than about 30° C., less than about 28° C., less than about 26° C., less than about 24° C., less than about 22° C., or less than about 20° C., inclusive of all values and ranges therebetween. In some embodiments, the temperatures of the first material, the second material, and/or the first mixture can be maintained at between about 20° C. and about 30° C., between about 20° C. and about 26° C., between about 21° C. and about 25° C., or between about 22° C. and about 24° C., inclusive of all values and ranges therebetween. In some embodiments, any of these temperature can be considered to be “room temperature” based upon climactic differences between different manufacturing locations. In some embodiments, the first material, second material, and/or the first mixture can be maintained at room temperature by incidental or intentional heating or cooling where climactic conditions in a particular manufacturing location would otherwise result in a material and/or mixture that is higher or lower than the values and ranges disclosed above.

In some embodiments, the initial viscosity of the first mixture after the combining 11 step can be less than about 25,000 cP, less than about 20,000 cP, less than about 15,000 cP, less than about 10,000 cP, less than about 8,000 cP, less than about 6,000 cP, less than about 4,000 cP, less than about 3,000 cP, less than about 2,000 cP, less than about 1,000 cP, less than about 900 cP, less than about 800 cP, less than about 700 cP, less than about 600 cP, less than about 500 cP, less than about 400 cP, less than about 300 cP, or less than about 200 cP, inclusive of all values and ranges therebetween. In some embodiments, the initial viscosity of the first mixture after the combining 11 step can be greater than about 200 cP, greater than about 300 cP, greater than about 400 cP, greater than about 500 cP, greater than about 600 cP, greater than about 700 cP, greater than about 800 cP, greater than about 900 cP, greater than about 1,000 cP, greater than about 2,000 cP, greater than about 3,000 cP, greater than about 4,000 cP, greater than about 5,000 cP, greater than about 6,000 cP, greater than about 8,000 cP, greater than about 10,000 cP, greater than about 15,000 cP, greater than about 20,000 cP, or greater than about 25,000 cP, inclusive of all values and ranges therebetween.

In some embodiments, the method 10 can comprise a sterilization step preceding the combining 11 step such that substantially no contaminant are incidentally introduced to the first mixture. In some embodiments, the sterilization step can comprise a chemical and/or thermal treatment of a processing vessel. In some embodiments, the processing vessel can comprise at least one of a mixing tank, a receiving vessel, spherical tank, columnar tank, underground storage tank, pressure vessel, atmospheric tank, mobile tank, rotating tank, chemical tank, or any other tank suitable for containing the first mixture before, during or after mixing.

The method 10 further comprises mixing the first mixture at room temperature 12. In some embodiments, mixing 12 can comprise a single stage of mixing for a duration or multiple stages of mixing. In some embodiments, the first mixture can be mixed until it is homogenized or it can be mixed to a degree of homogenization less than 100%. In some embodiments, the mixing 12 step can be carried out until the first mixture is less than about 99% homogenized, less than about 98% homogenized, less than about 97% homogenized, less than about 96% homogenized, less than about 95% homogenized, less than about 90% homogenized, less than about 85% homogenized, less than about 80% homogenized, less than about 75% homogenized, less than about 70% homogenized, less than about 60% homogenized, less than about 50% homogenized, less than about 40% homogenized, less than about 30% homogenized, less than about 20% homogenized, or less than about 10% homogenized, inclusive of all values and ranges therebetween.

In some embodiments, mixing 12 can be carried out using a stand mixer, high speed homogenizer mixers, static mixers, horizontal mixers, multi-shaft mixers, paddle mixers, drum mixers, high velocity mixers, agitators, dual cartridge mixers, metering mixers, mixing/dispensing machines, bench-top pint mixers, mounting lab mixers, large capacity industrial mixers, uniform shear field mixers, temperature control mixers, acoustic mixers, planetary mixers, double planetary mixers, kneading mixers, RAM mixers, mixers with low hydrodynamic shear stress mixing, open double helix design mixers, two component adhesive mixing systems, jiffy mixers, jiffler mixing blades, vortex mixer, auger blade mixers, mud mixers, wire mixers, mortar mixers, bucket mixers, two-part material mixers, two-part snuf-bak mixers, continuous flow mixers, and combinations thereof.

In some embodiments, mixing 12 can be carried out in a processing vessel of any size, comprising but not limited to greater than about 20 gallons, greater than about 50 gallons, greater than about 100 gallons, greater than about 200 gallons, greater than about 300 gallons, greater than about 400 gallons, greater than about 500 gallons, greater than about 750 gallons, greater than about 1,000 gallons, greater than about 1,250 gallons, greater than about 1,500 gallons, greater than about 2,000 gallons, greater than about 2,250 gallons, greater than about 2,500 gallons, greater than about 2,750 gallons, greater than about 3,000 gallons, greater than about 3,250 gallons, greater than about 3,500 gallons, greater than about 3,750 gallons, greater than about 4,000 gallons, greater than about 5,000 gallons, greater than about 7,500 gallons, greater than about 10,000 gallons, greater than about 50,000 gallons, or greater than about 100,000 gallons, inclusive of all values and ranges therebetween. In some embodiments, the processing vessel can be between about 20 gallons and about 100,000 gallons, between about 100 gallons and about 10,000 gallons, between about 200 gallons and about 7,500 gallons, between about 300 gallons and about 5,000 gallons, between about 500 gallons and about 4,000 gallons, between about 750 gallons and about 4,000 gallons, and between about 1,000 gallons and about 3,000 gallons, inclusive of all values and ranges therebetween.

In some embodiments, mixing 12 can be carried out for less than about two hours, less than about 90 minutes, less than about 75 minutes, less than about one hour, less than about 50 minutes, less than about 45 minutes, less than about 40 minutes, less than about 35 minutes, less than about 30 minutes, less than about 25 minutes, less than about 20 minutes, less than about 15 minutes, less than about 12 minutes, less than about 10 minutes, less than about 8 minutes, less than about 5 minutes, or less than about 2 minutes, inclusive of all value and ranges therebetween. In some embodiments, mixing 12 can be carried out for greater than about 2 minutes, greater than about 5 minutes, greater than about 8 minutes, greater than about 10 minutes, greater than about 12 minutes, greater than about 15 minutes, greater than about 20 minutes, greater than about 25 minutes, greater than about 30 minutes, greater than about 35 minutes, greater than about 40 minutes, greater than about 45 minutes, greater than about 50 minutes, greater than about one hour, greater than about 75 minutes, greater than about 90 minutes, or greater than about two hours, inclusive of all values and ranges therebetween.

The method 10 further comprises combining a third material and the first mixture at room temperature to form a second mixture 13. In some embodiments, the first mixture can mixed into the third material by communicating the first mixture from a first vessel into a second vessel, the second vessel containing the third material. In some embodiments, the third material can be mixed into the first mixture without communicating the first mixture out of the first vessel. Alternatively, in some embodiments, the first mixture and the third material can be communicated together into a new mixing vessel, e.g., by merging the two flow paths. In some embodiments, the first mixture and the third material can be at least partially mixed together before being communicated into the new mixing vessel. In some embodiments, this pre-mixing of the first mixture and the third material can be accomplished using a static (in-line) mixer, via inherent turbulence due to flow path tortuosity, through axial mixing, through longitudinal mixing, through laminar flow turbulence, using a Tee Mixer, using a coaxial jet mixer, or using any other suitable in-pipe mixing methods.

In some embodiments, the third material can comprise sodium hydroxide, sodium lauroamphoacetate, steareth-4, xanthan gum, benzyl alcohol, benzyl benzoate, benzyl cinnamate, benzyl salicylate, Cinnamal, citral, eugenol, garnesol, geraniol, isoeugenol, limonene, linaloolis, hydroxyethylcellulose, Lavandula angustifolia (lavender) oil, moringa oleifera seed oil, palmitamidopropyltrimonium chloride, panthenol, Passiflora edulis seed oil, phenoxyethanol, polyquaternium-37, potassium sorbate, ppg-1 trideceth-6, propylene glycol dicaprylate/dicaprate, Rosmarinus officinalis (rosemary) leaf extract, sodium hydroxide, sorbitan oleate, squalene, benzyl alcohol, benzyl benzoate, benzyl cinnamate, benzyl salicylate, cinnamal, citral, eugenol, farnesol, geraniol, isoeugenol, limonene, linalool, vetiver oil, orange oil, ylang oil, moringa oleifera, adasonia digitata seed, and/or amaranthus caudatus seed oils.

In some embodiments, the third material can be between about 0.01 wt % and about 99 wt % of the finished formulation, between about 1 wt % and about 90 wt %, between about 2 wt % and about 85 wt %, between about 3 wt % and about 80 wt %, between about 4 wt % and about 75 wt %, between about 5 wt % and about 70 wt %, between about 6 wt % and about 65 wt %, between about 7 wt % and about 60 wt %, between about 8 wt % and about 55 wt %, between about 9 wt % and about 50 wt %, between about 10 wt % and about 45 wt %, between about 11 wt % and about 40 wt %, between about 12 wt % and about 35 wt %, between about 13 wt % and about 30 wt %, between about 14 wt % and about 25 wt %, between about 15 wt % and about 20 wt %, between about 1 wt % and about 50 wt %, between about 1 wt % and about 25 wt %, between about 1 wt % and about 20 wt %, between about 20 wt % and about 99 wt %, between about 25 wt % and about 98 wt %, between about 30 wt % and about 97 wt %, between about 35 wt % and about 96 wt %, between about 40 wt % and about 95 wt %, between about 45 wt % and about 94 wt %, between about 50 wt % and about 93 wt %, between about 55 wt % and about 92 wt %, between about 60 wt % and about 91 wt %, between about 65 wt % and about 90 wt %, between about 70 wt % and about 85 wt %, and about 75 wt % to about 85 wt %, inclusive of all values and ranges therebetween.

The method 10 further comprises mixing the second mixture at room temperature 14. In some embodiments, mixing 14 can be carried out using a substantially similar method, substantially similar equipment, and/or a substantially similar mixing duration as described above with regard to mixing 12. Alternatively, in some embodiments, mixing 14 can be carried out at a larger scale and therefore mixing of the third material and the second mixture may be carried out for a longer duration, at a slower mixing speed, and/or using a different mixer than for mixing 12.

The method 10 further comprises combining a fourth material and the second mixture at room temperature to form a third mixture 15. In some embodiments, the fourth material can be communicated into a mixing vessel containing the second mixture. Alternatively, in some embodiments, the fourth material and the second mixture can be communicated together into a new mixing vessel, e.g., by merging the two flow paths. In some embodiments, the fourth material and the second mixture can be at least partially mixed together before being communicated into the new mixing vessel. In some embodiments, this pre-mixing of the fourth material and the second mixture can be accomplished using a static (in-line) mixer, via inherent turbulence due to flow path tortuosity, through axial mixing, through longitudinal mixing, through laminar flow turbulence, using a Tee Mixer, using a coaxial jet mixer, or using any other suitable in-pipe mixing methods.

In some embodiments, the fourth material can comprise myristamidopropyl betaine, sodium benzoate, tetrasodium glutamate diacetate, trisodium ethylenediamine disuccinate, benzoic acid, benzyl alcohol, propylene glycol, sodium benzoate, and/or basil root extract.

In some embodiments, the fourth material can be between about 0.01 wt % and about 99 wt % of the finished formulation, between about 1 wt % and about 90 wt %, between about 2 wt % and about 85 wt %, between about 3 wt % and about 80 wt %, between about 4 wt % and about 75 wt %, between about 5 wt % and about 70 wt %, between about 6 wt % and about 65 wt %, between about 7 wt % and about 60 wt %, between about 8 wt % and about 55 wt %, between about 9 wt % and about 50 wt %, between about 10 wt % and about 45 wt %, between about 11 wt % and about 40 wt %, between about 12 wt % and about 35 wt %, between about 13 wt % and about 30 wt %, between about 14 wt % and about 25 wt %, between about 15 wt % and about 20 wt %, between about 1 wt % and about 50 wt %, between about 1 wt % and about 25 wt %, between about 1 wt % and about 20 wt %, between about 20 wt % and about 99 wt %, between about 25 wt % and about 98 wt %, between about 30 wt % and about 97 wt %, between about 35 wt % and about 96 wt %, between about 40 wt % and about 95 wt %, between about 45 wt % and about 94 wt %, between about 50 wt % and about 93 wt %, between about 55 wt % and about 92 wt %, between about 60 wt % and about 91 wt %, between about 65 wt % and about 90 wt %, between about 70 wt % and about 85 wt %, and about 75 wt % to about 85 wt %, inclusive of all values and ranges therebetween.

The method 10 further comprises mixing the third mixture to form the personal hygiene product 16. In some embodiments, mixing 16 can be carried out using a substantially similar method, substantially similar equipment, and/or a substantially similar mixing duration as described above with regard to mixing 12. Alternatively, in some embodiments, mixing 14 can be carried out at a larger scale and therefore mixing of the third material and the second mixture may be carried out for a longer duration, at a slower mixing speed, and/or using a different mixer than for mixing 12. In some embodiments, final formulation of the PHP can be carried out by mixing 16 the third mixture for greater than about 5 minutes, greater than about 10 minutes, greater than about 20 minutes, greater than about 30 minutes, greater than about 40 minutes, greater than about 50 minutes, or greater than about one hour, inclusive of all values and ranges therebetween. In some embodiments, final formulation of the PHP can comprise the addition of finishing additives comprising but not limited to acidifiers, pearlescent additives, surfactants, and thickeners.

In some embodiments, in addition to the materials disclosed above, the PHP formulation can also comprise greater than about 0.001 wt % of ammonium chloride, ammonium lauryl sulfate, glycol, sodium laureth sulfate, sodium lauroamphoacetate, coconut oils, polysorbate 20, polysorbate 80, glycol-based surfactants, PEG-150, citric acid, quaternium-15, polyquaternium-10, Di-PPG-2 myreth-10 adipate, fungicides, ketoconazole, zinc pyrithione, selenium sulfide, coal tar, salicylate derivatives, tea tree oil, colorants, Triticum vulgare derivatives, Hordeum vulgare derivatives, secale cereal derivatives, Avena sativa derivatives, tocopherol/tocopheryl acetate, hydrolyzed wheat protein, hydrolyzed wheat starch, cyclodextrin, amino peptide complex, maltodextrin, dextrin, dextrin palmitate, hydrolyzed malt extract, phytophoingosine extract, prolamine, beta glucan, disodium wheat germamido PEG-2-sulfosuccinate, fermented grain extract, AMP-Isostearoyl, PG-propyl silanetriol, PVP crosspolymer, Ethyldimonium ethosulfate, yeast extract, phytospingosine extract, polyethoxylated synthetic glycolipids, polyethoxylated synthetic monoglycerides, long-chain aliphatic fatty acids, cationic polyelectrolyte polymers, humectants, almond oil, hydrolyzed protein, silicones, dimethicone, cyclomethicone, essential fatty acids, panthenol, benzophenone-4, ethylhexyl methoxycinnamate, cinnamidopyltrimonium chloride, cetyl alcohol, grape seed oil, alkylbenzenesulfonates, lignin sulfonates, fatty alcohol ethoxylates, alkylphenol ethoxylates, sodium dodecyl sulfate, cetrimonium bromide, Triton X-100, Triton X-114, octyl thioglucoside, octyl glucoside, dodecyl maltoside, docusate, dioctyl sodium sulfosuccinate, perfluorooctanesulfonate, perfluorobutanesulfonate, alkyl-aryl ether phosphates, alkyl ether phosphates, carboxylates, sodium stearate, sodium lauroyl sarcosinate, perfluorononanoate, perfluorooctanoate, octaethylene glycol monododecyl ether, pentaethylene glycol monododecyl ether, polyethoxylated tallow amine, cocamide monethanolamine, cocamide diethanolamine, poloxamers, glycerol monstearate, clycerol monlaurate, nonoxynols, sorbitan monolaurate, sorbitan monostearate, sorbitan tristearate, Tween 20, Tween 40, Tween 60, Tween 80, Decyl glucoside, phosphine oxide, calcium chloride, calcium disodium ethylene diamine tetra-acetate, glucono delta-lactone, sodium gluconate, potassium gluconate, sodium tripolyphosphate, sodium hexametaphosphate, ethylenediaminetetraacetic acid, behentrimonium chloride, cocamidopropyl betaine, esters of phosphoric acid, polyethylene glycol esters, polyols, ethoxylated amines, glycerol esters, glycerol monostearate, sorbic acid, sodium sorbate, benzoic acid, sodium benzoate, hydroxybenzoate, sulfur dioxide, sulfites, nitrite, nitrate, lactic acid, propionic acid, sodium propionate, gallic acid, sodium gallate, tocopherols, other suitable additives, or combinations thereof.

In some embodiments, once the large volume PHP is formulated (e.g., according to the method 10), the PHP formulation can be filtered. In some embodiments, the filtration process can comprise passing the finished PHP formulation through a high flux screen, membrane, filter, exchange media, centrifuge, settling tank, aeration tank, or any other process suitable for removing contaminants from the finished PHP formulation.

In some embodiments, once the large volume PHP has been filtered, sub-volumes of the PHP formulation can be dispensed into consumer packaging (e.g., bottles). In some embodiments, the consumer packaging can be any suitable material comprising but not limited to plastic, glass, cardboard, rubber, metal, or wood. In some embodiments, each consumer packaging container can have a uniform capacity to facilitate automated dispensing of the PHP formulation into the consumer packaging container. In some embodiments, the consumer packaging container can have an open end and a closed end such that it can be filled from the open end and a cap, seal, lid, or other closure can be applied once the container is filled with the PHP formulation. In some embodiments, the consumer packaging container can be pre-decorated, can be decorated once filled with the PHP formulation, or can be decorated once the container has been sealed. In some embodiments, the individualized containers of PHP formulation can then be further packaged into a box and onto a crate for distribution.

In some embodiments, the room temperature PHP formulation makes it easier to pre-decorate the consumer packaging container than when the conventional PHP formulation method is used. Without wishing to be bound by any particular theory, the higher temperature PHP formulation produced according to conventional methods may cause deterioration of decoration, may cause the distortion or deformation of the container itself, may cause overpressurization of the container once sealed due to thermal expansion of the gaseous atmosphere in the headspace of the container, and/or may complicate handling of the bottles due to deterioration of secondary packaging such as cardboard boxes, shrink-wrapping used to secure the containers on a crate, and other packaging problems.

### Working Example 1

A first formulation was made by mixing together water, sodium lauroyl methyl isethionate, peg-150 pentaerythrityl tetrastearate sodium cocoyl apple amino acids, polysorbate 20, and lauramidopropyl betaine using a propeller mixer at a maximum mixer speed of about 300 rpm.

A second formulation was made by slowly mixing together fragrance (parfum), glycerin, ppg-2 hydroxyethyl cocamide, acrylates copolymer, adansonia digitata seed oil, amaranthus caudatus seed oil, cananga odorata flower oil, caprylyl glycol, cocamide mipa, disodium edta, glycol distearate, Helianthus annuus (sunflower) seed oil, hexylene glycol, Lavandula angustifolia (lavender) oil, menthyl ethylamido oxalate, moringa oleifera seed oil, panthenol, phenoxyethanol, potassium sorbate, and Rosmarinus officinalis (rosemary) leaf extract with the first formulation.

A third formulation was made by mixing sodium hydroxide, sodium lauroamphoacetate, steareth-4, xanthan gum, benzyl alcohol, benzyl benzoate, benzyl cinnamate, benzyl salicylate, Cinnamal, citral, eugenol, farnesol, geraniol, isoeugenol, limonene, and linaloolis.

A fourth formulation was made by mixing myristamidopropyl betaine, sodium benzoate, tetrasodium glutamate diacetate, and trisodium ethylenediamine disuccinate.

The third formulation and fourth formulation were slowly mixed together in a vessel until substantially combined to form a fifth formulation.

The third formulation and the fifth formulation were mixed together slowly for about 30 minutes until the solution becomes a thick and clear gel, the sixth formulation.

Sodium chloride was added to the sixth formulation to adjust viscosity and citric acid was added to adjust the pH to about 5.5.

The viscosity and pH-adjusted sixth formulation can be a hair shampoo, which can then be charged to individual consumer packaging containers.

### Working Example 2

A first formulation was made by mixing together water, hydroxypropyl starch phosphate, caprylic/capric/myristic/stearic triglyceride, glycerin, fragrance (parfum), isoamyl laurate, caprylic/capric triglyceride, hydrogenated vegetable oil, polysorbate 60, trimethylolpropane tricaprylate/tricaprate, and acetamidopropyl trimonium chloride using a propeller mixer at a maximum mixer speed of about 300 rpm.

A second formulation was made by slowly mixing together adansonia digitata seed oil, amaranthus caudatus seed oil, cananga odorata flower oil, caprylyl glycol, cetrimonium chloride, citric acid, Crambe abyssinica seed oil, decyl glucoside, dicaprylyl ether, disodium edta, ethylhexyl olivate, glyceryl oleate, guar hydroxypropyltrimonium chloride, Helianthus annuus (sunflower) seed oil, hexylene glycol, hydrogenated castor oil/sebacic acid copolymer, hydrolyzed silk, and hydrolyzed soy protein with the first formulation.

A third formulation was made by mixing hydroxyethylcellulose, Lavandula angustifolia (lavender) oil, moringa oleifera seed oil, palmitamidopropyltrimonium chloride, panthenol, Passiflora edulis seed oil, phenoxyethanol, polyquaternium-37, potassium sorbate, ppg-1 trideceth-6, propylene glycol dicaprylate/dicaprate, Rosmarinus officinalis (rosemary) leaf extract, sodium hydroxide, sorbitan oleate, squalene, benzyl alcohol, benzyl benzoate, benzyl cinnamate, benzyl salicylate, cinnamal, citral, eugenol, farnesol, geraniol, isoeugenol, limonene, and linalool.

A fourth formulation was made by mixing benzoic acid, benzyl alcohol, propylene glycol, and sodium benzoate.

The third formulation and fourth formulation were slowly mixed together in a vessel until substantially combined to form a fifth formulation.

The third formulation and the fifth formulation were mixed together slowly for about 30 minutes until the solution becomes a thick and creamy consistency, the sixth formulation, a hair conditioner, which was then charged to individual consumer packaging containers.

### Working Example 3

A first formulation was made by mixing together C12-C15 alkyl benzoate (benzoic acid and long-chain alcohols) and isostearyl linoleate using a propeller mixer at a maximum mixer speed of about 300 rpm.

A second formulation was made by slowly mixing together sunflower oil and soybean oil with the first formulation.

A third formulation was made by slowly mixing together aloe and calendula in sunflower oil with the second formulation.

A fourth formulation was made by slowly mixing together vetiver (chrysopogon zizanioides) oil, orange oil, ylang (cananga odorata) oil, moringa oleifera, adasonia digitata seed, and amaranthus caudatus seed oil with the third formulation.

A fifth formulation was made by slowly mixing together basil root extract with the fourth formulation. The fifth formulation was mixed for about 20 minutes, filtered to remove contaminants and any agglomerations, and then charged to individual consumer packaging containers.

The indefinite articles “a” and “an,” as used herein, unless clearly indicated to the contrary, should be understood to mean “at least one.”

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally comprising elements other than B); in another embodiment, to B only (optionally comprising elements other than A); in yet another embodiment, to both A and B (optionally comprising other elements); etc.

As used herein, “or” should be understood to have the same meaning as “and/or” as defined above. For example, when separating items in a list, “or” or “and/or” shall be interpreted as being inclusive, i.e., the inclusion of at least one, but also comprising more than one, of a number or list of elements, and, optionally, additional unlisted items. Only terms clearly indicated to the contrary, such as “only one of” or “exactly one of,” or, when used in claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in claims, shall have its ordinary meaning as used in the field of patent law.

As used herein, the phrase “at least one,” in reference to a list of one or more elements, should be understood to mean at least one element selected from any one or more of the elements in the list of elements, but not necessarily comprising at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally comprising more than one, A, with no B present (and optionally comprising elements other than B); in another embodiment, to at least one, optionally comprising more than one, B, with no A present (and optionally comprising elements other than A); in yet another embodiment, to at least one, optionally comprising more than one, A, and at least one, optionally comprising more than one, B (and optionally comprising other elements); etc.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±20%, in another example ±10%, in another example ±5%, in another example ±1%, and in yet another example ±0.1% from the specified amount, as such variations are appropriate to perform the disclosed method.

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

All examples and/or embodiments are deemed to be non-limiting throughout this disclosure. Also, no inference should be drawn regarding those embodiments discussed herein relative to those not discussed herein other than it is as such for purposes of reducing space and repetition. For instance, it is to be understood that the logical and/or topological structure of any combination of any data flow sequence(s), program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure. Furthermore, it is to be understood that such features are not limited to serial execution, but rather, any number of threads, processes, processors, services, servers, and/or the like that may execute asynchronously, concurrently, in parallel, simultaneously, synchronously, and/or the like are also contemplated by the disclosure. As such, some of these features may be mutually contradictory, in that they cannot be simultaneously present in a single embodiment. Similarly, some features are applicable to one aspect of the innovations, and inapplicable to others. In addition, the disclosure includes other innovations that are disclosed and may not explicitly recited. As such, it should be understood that advantages, embodiments, examples, functional, features, logical, operational, organizational, structural, topological, and/or other aspects of the disclosure are not to be considered limitations on the disclosure as defined by the embodiments, examples, claims or limitations on equivalents to the embodiments, examples, and/or claims. It is to be understood that, depending on the particular needs and/or characteristics of a PHP, various embodiments or portions of various embodiments of the PHP may be implemented that allow a great deal of flexibility and customization. For example, aspects of the PHP may be changed to allow for a PHP that is gluten-free, for a “no tears” formulation, for a hair dye-tolerant shampoo, etc. While various embodiments and discussions of the PHP have been directed to shampoo, conditioner, and scalp detoxifying formulations, however, it is to be understood that the embodiments described herein may be readily configured and/or customized for a wide variety of other applications and/or implementations.

1. A method of manufacturing a personal hygiene product, comprising:
combining a first material and second material at room temperature to form a first mixture, the first material including at least two of: sodium lauroyl methyl isethionate, PEG-150 pentaerythrityl tetrastearate, sodium cocoyl apple amino acids, polysorbate 20, lauramidopropyl betaine, water, hydroxypropyl starch phosphate, caprylic/capric/myristic/stearic triglyceride, glycerin, parfum, isoamyl laurate, caprylic/capric triglyceride, hydrogenated vegetable oil, polysorbate 60, trimethylolpropane tricaprylate/tricaprate, acetamidopropyl trimonium chloride, C12-C15 alkyl benzoates, isostearyl linoleate, sunflower oil, and./or soybean oil; and the second material including at least two of: parfum, glycerin, PPG-2 hydroxyethyl cocamide, acrylates copolymer, adansonia digitata seed oil, amaranthus caudatus seed oil, cananga odorata flower oil, caprylyl glycol, cocamide mipa, disodium edta, glycol distearate, Helianthus annuus seed oil, hexylene glycol, Lavandula angustifolia oil, menthyl ethylamido oxalate, moringa oleifera seed oil, panthenol, phenoxyethanol, potassium sorbate, Rosmarinus officinalis leaf extract, adansonia digitata seed oil, amaranthus caudatus seed oil, cananga odorata flower oil, caprylyl glycol, cetrimonium chloride, citric acid, Crambe abyssinica seed oil, decyl glucoside, dicaprylyl ether, disodium edta, ethylhexyl olivate, glyceryl oleate, guar hydroxypropyltrimonium chloride, hexylene glycol, hydrogenated castor oil/sebacic acid copolymer, hydrolyzed silk, hydrolyzed soy protein, isostearyl linoleate, sunflower oil, soybean oil, aloe, and/or calendula in sunflower oil;
mixing the first mixture at room temperature;
combining a third material and the first mixture at room temperature to form a second mixture, the third material including at least two of: sodium hydroxide, sodium lauroamphoacetate, steareth-4, xanthan gum, benzyl alcohol, benzyl benzoate, benzyl cinnamate, benzyl salicylate, cinnamal, citral, eugenol, farnesol, geraniol, isoeugenol, limonene, linaloolis, hydroxyethylcellulose, Lavandula angustifolia oil, moringa oleifera seed oil, palmitamidopropyltrimonium chloride, panthenol, Passiflora edulis seed oil, phenoxyethanol, polyquaternium-37, potassium sorbate, PPG-1 trideceth-6, propylene glycol dicaprylate/dicaprate, Rosmarinus officinalis leaf extract, sodium hydroxide, sorbitan oleate, squalene, benzyl alcohol, benzyl benzoate, benzyl cinnamate, benzyl salicylate, cinnamal, citral, eugenol, farnesol, geraniol, isoeugenol, limonene, linalool, aloe, calendula in sunflower oil, vetiver oil, orange extract, ylang oil, moringa oleifera, adasonia digitata seed, and amaranthus caudatus seed oil;
mixing the second mixture at room temperature;
combining a fourth material and the second mixture at room temperature to form a third mixture, the fourth material including at least two of myristamidopropyl betaine, sodium benzoate, tetrasodium glutamate diacetate, trisodium ethylenediamine disuccinate, benzoic acid, benzyl alcohol, propylene glycol, sodium benzoate, vetiver oil, orange extract, ylang oil, moringa oleifera, adasonia digitata seed, amaranthus caudatus seed oil, and basil root extract;
mixing the third mixture at room temperature, wherein substantially no thermal energy is added to the first material, the second material, the third material, and the fourth material during a compounding process.

2. The method of claim 1, wherein substantially no thermal energy is added to the first material, the second material, the first mixture, the third material, the second mixture, the fourth material, and the third mixture.

3. The method of claim 1 or claim 2, wherein the personal hygiene product is at least one of a hair shampoo, a hair conditioner, and a scalp detoxifier.

4. The method of claim 1, claim 2, or claim 3, wherein mixing the first mixture, mixing the second mixture, and mixing the third mixture includes using a propeller mixer.

5. The method of claim 4, wherein the propeller mixer is rotated at a speed of greater than about 200 rpm.

6. The method of any one of the preceding claims, further comprising adjusting a pH of the third mixture to between about 4.5 and about 6.5 by adding a quantity of an acidifier.

7. The method of any one of the preceding claims, further comprising adjusting a viscosity of the third mixture to between about 100 cP and about 25,000 cP by adding a quantity of a thickener.

8-17. (canceled)
